(19) United States
(12) Patent Application Publication
(10) Pub. No.: US 2003/0176763 A1
(43) Pub. Date: Sep. 18, 2003

(54) Slow release device for controlled delivery of liquid material

(76) Inventor: Nachman Eckstein, Tel Aviv (IE)

Correspondence Address: FOGG AND ASSOCIATES, LLC, P.O. BOX 581339, MINNEAPOLIS, MN, 55458-1339, US

(21) Appl. No.: 10/333,436

(22) PCT Filed: Jul. 9, 2001
(86) PCT No.: PCT/IL01/00625

(30) Foreign Application Priority Data
Jul. 20, 2000 (IL) 137431

(51) Int. Cl. A61B 17/43

(52) U.S. Cl. 600/35

(57) ABSTRACT

A sterilizable, compact, portable device for dispensing predetermined amount, at a predetermined flow rate, of a selected liquid preparation into a specific target-organ or tissue in a mammalian body, in particular, of a human. Accessories may also be included for assisting in placing, and/or fixing, the device in right position within, or in adjacent to, the desired organ.

### FIELD OF THE INVENTION

The present invention relates to a self-contained, disposable, inexpensive, sterilizable, prolonged-release system, applicable in slow and controllable delivery of a selected liquid material. More specifically, the subject of the present invention is a new device useful for prolonged release of a selected material into specific organs and/or cavities of a mammal body, including a human. A major advancement of said device is based on a controllable process resulted in gas generation by a reaction of one or more reagents of which reagents at least one is in an encapsulated form.

### BACKGROUND OF THE INVENTION

Various slow-pumping devices useful for prolonged, slow-release of selected material, in a suspension form, into specific organs of mammals, in particular, human body, are known.

Several major mechanisms of prolonged, slow-delivery are recognized—among them are:

1. Generated osmotic pressure—driven delivery (osmotic pumps).

2. Release of active material from a core by means of contact with a surrounding liquid (liposomal drug delivery).

3. Generated pulses—driven delivery (syringe pamps).

4. Generated gas—driven delivery.

Among the best known prolonged, slow-release systems are the devices for artificial insemination and for delivery of biological active materials (in particular, medicaments) into specific organs.

Various slow-release artificial insemination devices are known, among them are:

U.S. Pat. No. 5,536,243 reveals a self-contained prolonged time-release artificial insemination device introducing a bolus of semen into the cervical canal or uterus over a period of hours. The device includes a cervical cap adapted to conform and adhere to the cervix and includes an elongated nipple that extends in a perpendicular direction from the cap for insertion into the cervical canal or uterus. A time-release mechanism is provided in communication with the nipple for delivering semen through the nipple and includes a tubular body, which defines a semen chamber and expansion chamber. Within the expansion chamber, a quantity of water-swellable material is disposed for absorbing water from the reservoir and expanding so that the plunger is urged towards the cervical cap and the semen is discharged through the nipple and into the cervix or uterus over a period of hours.

U.S. Pat. No. 5,562,654 provides an apparatus for a prolonged time-released delivery of selected preparations, for various purposes, including artificial insemination, into a patient's uterine cavity. The apparatus includes an osmotic pump for expelling the preparation over time and a catheter for delivery the expelled material into the uterine cavity. An inflatable balloon on the catheter holds the apparatus in position in the patient. The osmotic pump contains an inner reservoir chamber for holding a pre-determined amount of the selected preparation, subjected for a prolonged time release and delivery, and an outer chamber for holding an osmotically active agent. The inner and outer chambers are separated by an impermeable membrane. Water from vaginal secretions passes through a semi-permeable membrane and enters the outer chamber that holds the osmotically active agent causing that agent to swell. The entering water and resulting swelling increase the pressure in the outer chamber pressing upon the impermeable membrane of the inner reservoir chamber. As the pressure slowly builds, the impermeable membrane compresses the inner reservoir chamber, thereby slowly expelling sperm cell suspension from the chamber into a lumen, from which it is discharged into the uterine cavity.

U.S. Pat. No. 5,904,665 discloses an intrauterine, automated prolonged slow release, artificial insemination method in which motile sperm are released into the uterus in an organized, programmed procedure, over an extended period of time. A catheter, attachable to an external pumping means, provided with an inflatable balloon, and attachable to a pumping means, is inserted into the uterine cavity and aliquots of spermatozoa containing medium are injected at a rate of 10 to 20 mm/hr (approximately every 30 seconds) for between 4 to 6 hours. Each aliquots contains 8,000 to 75,000 motile sperm. The pumping means (such as pulsing syringe pump) delivers the aliquots of sperm containing medium through the delivery channel, into the uterus.

Various slow-release drug delivery systems are known, among them are liposomal-releasing systems and prolonged delivery systems that are driven by a generated osmotic-pressure or by a generated-gas:

U.S. Pat. No. 5,242,406 provides a liquid (including drugs) delivery device containing a first contractible-chamber on one side of a diaphragm for holding a supply of the liquid to be delivered, and a second contractible-chamber on the opposite side of the diaphragm including an electrolytic cell capable of generating a gas according to the electrical current passed through the cell electrolyte. The device further includes a second diaphragm, a control valve, and a spring, for compensating the rate of delivery of the liquid for variations in ambient pressure and temperature.

U.S. Pat. No. 5,785,688 describes an apparatus useful for subcutaneous drug delivery includes a fluid reservoir disposed within a housing for storing the fluid, a pump or pressurized chamber for pressurizing a driving gas and exerting a force on the fluid reservoir to expel the fluid content, and a needle communicating with the reservoir.

U.S. Pat. No. 5,800,420 teaches a liquid delivery device comprising a reservoir and a gas generation chamber therein separated by a displaceable membrane. Gas generated by an electrolytic cell under the control of a microprocessor causes the gas generation chamber to expand and the reservoir to contract, thereby discharging the liquid drug from the reservoir via a needle into the skin.

U.S. Pat. No. 5,840,332 reveals a gastrointestinal delivery system which comprises a drug in combination with a core material. The core material is surrounded by a water-insoluble coating material in which particulate water-insoluble material is embedded. When the delivery device inserted into the gastrointestinal tract, the particulate matter takes up liquid, thus forming channels that allow the slow release of drug from the core into the gastrointestinal tract.

U.S. Pat. No. 5,848,991 provides an intradermal liquid drug delivery device to a subject via the skin. The device contains an expansible-contractible chamber which is expanded upon being filled with the drug and contracted to dispense the drug. The device permits delivery of drugs of relatively large molecular size and at a slow rates which can be precisely controlled, applying means for activity discharging the drug from the drug reservoir. The means for actively discharging the drug comprises a plurality of electrically controlled gas generators (electrolytic cells) within the device for generating gas to separately contract the plurality drug reservoirs in order to discharge the drug therefrom.

U.S. Pat. No. 5,858,001 discloses a cartridge-based liquid drug delivery device. A delivery needle, in communication with the interior of the cartridge, penetrates the ski of the subject while delivers the liquid drug. This action also causes the actuation of a citric acid/sodium bicarbonate gas generator, which generates a gas (CO₂) to move a piston within the cartridge, compressing the drug compartment. This compression causes a stopper to be penetrated by a conduit in communication with the delivery needle, allowing the drug to be ejected from the compartment through the needle and into the subcutaneous tissue of the subject.

U.S. Pat. No. 5,904,934 relates to a ruminal drug delivery device comprising a semi-permeable membrane defining a compartment containing a swellable osmotic agent expandable driving member, a drug to be dispensed, and a density element. The device contains a gas-impermeable barrier means that separates the density element from the other components within the delivery device for isolating gases evolved from the density element from the other components within the device.

Neither of the systems described above teaches a prolonged-release device applicable in either insemination and/or drug-delivery procedures that is based on a gas-generating reaction in which at least one of the reactive ingredients is in an encapsulated form.

Furthermore, systems described above suffer from some major disadvantages, among them are:

1. Systems generating gas by means of electrolysis, may provide at the same time some undesired by-products and/or physiological effects.

2. Rate of osmotic-pressure builds-up is difficult to control since it depends upon diffusion of water from surrounding vaginal (or other organs) secretions.

Consequently, there is a need to provide a well-controlled, self-contained, prolonged-release device applicable in a slow delivery of a selected liquid material that will be free of the above mentioned drawbacks.

It is an object of the present invention to provide a new self-contained, prolonged release, compact, disposable, sterilizable, inexpensive, portable device for dispensing into a specific target-organ or tissue in a mammalian body, and in particular in a human, of a predetermined amount of a selected liquid preparation, at a predetermined flow rate, comprising:

(I) a rigid housing;

(II) an inner non-stretchable fluid reservoir chamber (A) containing a predetermined amount of a selected fluid preparation intended for discharging under compression;

(III) an inner non-stretchable gas-generation chamber (B) containing at least one ingredient in an encapsulated form, which may react in a reaction yielding a gas;

(IV) an impermeable, stretchable elastomeric diaphragm, separating said chambers (A) and (B); provided that the evolving gas in chamber (B) exerting a pressure on the expansible diaphragm, which compresses the fluid reservoir compartment (A), resulted in expelling the fluid content from chamber (A);

(V) a tubule, a conduit or a hollow needle having an inner end which communicates with chamber (A) and an outer end which projects outwardly of the housing a short distance, used for delivering the expelled liquid from the reservoir chamber (A) into the target-organ or tissue in the mammal body;

(VI) inlets for inserting the fluid preparation into chamber (A) and water or other solvent into chamber (B)

(VII) optionally accessories for assisting in placing, and/or fixing, the device in right position within, or in adjacent to, the desired organ.

It is a further object of present invention to provide a new device applicable in insemination and fertilization of a mammal, and in particular of a human being, that is administered into the vagina and positioned adjacent to the cervix. The device intends to release aliquots of processed semen into the uterus at a controllable, prolonged, slow rate.

It is yet another object of present invention to provide a device applicable in a prolonged, slow release of a medicament into a target-organ in a mammalian body, in particular in a human body.

### SUMMARY OF THE INVENTION

The device of present invention is sterilizable, self-contained, slow-release, applicable in prolonged delivery of a desired liquid material at a well-controlled rate, and it operates independently of the surrounding environment. Such a device is ideal for insemination and fertilization processes. The device which is inserted into the vagina and placed adjacent to the cervix acting in an incubation conditions of about 37° C.

It is well recognized that difficulties in conception may originate in low sperm count and in inadequate flow of semen to the vicinity of the ovaries. Intrauterine insemination (IUI) is being done today in a manual way through the cervix using a syringe with the processed semen solution (bolus technique). Slow release insemination improves the percentage of pregnancy with a better possibility for the connection between the semen and the ovulation egg. Statistically speaking, it was demonstrated that slow insemination improves the pregnancy percentage. Furthermore, in the bolus technique a large volume of the semen solution is injected into the uterus cavity and then move toward the fallopian tube, such rapid injection of the solution creates a possibility of removing the egg with the semen solution back to the fallopian tube. This possibility is avoided by using slow release insemination.

In order to increase the yield and productivity of conception, various artificial insemination means and devices were developed using processed semen obtained from the spouse. More specifically, semen suspensions are delivered into the uterus or into the cervix canal using an external syringe pump or other delivery means. External syringe pumps that provides homogenous semen suspensions under controllable temperature are expensive and require the women to lie on the physician bed for several hours in an uncomfortable position. Thus, using the device of present invention (FIG. 1) freeing the women of the above limitations and at the same time provides economical benefits as well as comfort, easy to use and high yield of efficacy and productivity.

The device of present invention contains at least two chambers (FIGS. 2 & 3): chamber B (P2), hermetically sealed, contains the particulate reactive ingredients of which at least one is in encapsulated form, and chamber A (P1) contains the semen suspension or medicament liquid formulation. As reaction is triggered, gas is generated in chamber (B), followed by expansion of the elastic diaphragm (P3) serves as partition between chambers (A) and (B). As a result, the fluid preparation is pushed from chamber (A) through an outlet tubule towards the desired organ or tissue. The device contains optional accessories assisting in placing it in right position within, or in adjacent to, the desired organ. For example, for an artificial insemination device the accessories parts include a tube (P5) and a piston (P6) used for administrating (pushing) the device into the vagina and a stick (P7) and a “V” guide (P8) for locating the tubule tip in the optimal position. Furthermore, the device may contain external adjustable projections for fixing it and avoiding undesired movements. In addition, a string is attached to its end for its removal after use.

The device contains inlets for inserting the fluid preparation into chamber (A) and water, or other solvent, into chamber (B). For example, the fluid preparation (including the semen sample) may be injected into chamber A through a rubber (or silicon) septum by a plastic syringe (FIG. 4). Water (or any other solvent) may be injected into chamber B through a rubber septum, as well.

The device may be in any desired shape that fits and/or compatible with the target-organ, resulting in discharging the liquid preparation to the right location or site. In a preferred embodiment of present invention, it is made of a rigid or flexible, transparent or opaque sterilizable, non-toxic polymeric material. For example, the chambers A and B are optionally made of polyethylene; the impermeable membrane (diaphragm) is optionally made of thermoplastic elastomer; the cup is optionally made of polyacetate; the tube is optionally made of polyethylene; the piston and the stick are optionally made of high-density polyethylene and the “V” guide is optionally made of thermoplastic elastomer.

Processes for encapsulation are well recognized in the pharmaceutical and food industries. The fluidized bed process is well known for its drying efficiency, and is used for coating fluidized particles by a variety of techniques. The encapsulation process used in the present invention is the Wurster process (known also as Wurster system) which is based on an air suspension technique. The Wurster coating technique is well suited to uniformly coat or encapsulate individual particulate materials. It is characterized by the location of a spray nozzle at the bottom of a fluidized bed of solid particles. The particles are suspended in the fluidizing air stream that is designed to induce a cyclic flow of the particles past the spray nozzle. The nozzle sprays an atomized flow of coating solution, suspension, or other coating vehicle. The atomized coating material collides with the particles as they are carried away from the nozzle. The temperature of the fluidizing air is set to appropriately evaporate solution or suspension solvent or solidify the coating material shortly after colliding with the particles. All coating solids are left on the particles as a part of the developing film or coating. This process is continued until each particle is coated uniformly to the desired film thickness. The Wurster process is an industry recognized coating technique for precision application of film coat to particulate materials such as powders, crystals or granules. The technology is capable of encapsulating solid materials with diameters ranging from near 50 μm to several cm.

In accordance with a preferred embodiment of present invention, a capsule comprising two coating layers was prepared. More specifically, the capsule is made of an inert core (for example, a spherical sugar particle) which is isolated by a first coating layer (initial coating) to prevent the water from dissolving the core material. The initial coating layer may contain, for example, a mite of Ethocel (ethyl cellulose) and stearic acid. The coated core is further encapsulated with a second coating layer (main coating) comprising a mixture of at least one reactive ingredient (for example calcium carbonate) and an inert cellulose derivative(s) such as, for example, Ethocel and Klucel (hydroxypropyl cellulose). Upon administration of water (or any other suitable solvent) into chamber B, the water dissolve the non-encapsulated particulate reagent(s) that may be present in chamber B (for example, an acid), followed by slowly penetration into the capsule, dissolving the encapsulated reactant(s) (for example, a carbonate) resulted in activation of the gas-producing reaction.

### BRIEF DESCRIPTION OF THE DRAWINGS

FIG. 1: The device of present invention is positioned within the vagina, adjacent to the cervix. The projected tubule is in proximity to the fallopian tubes;

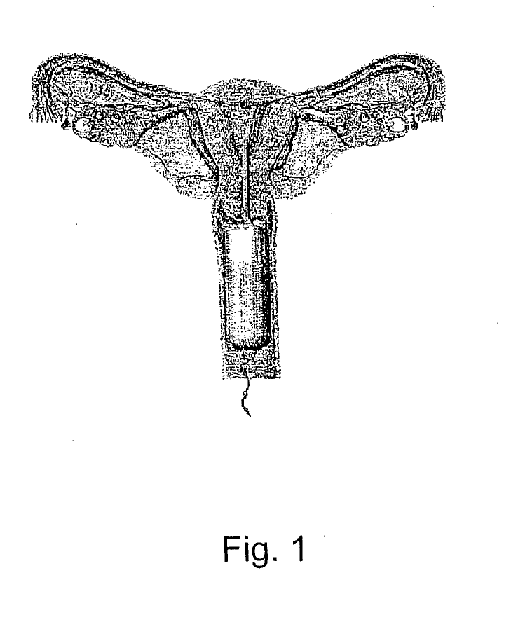

FIG. 2: demonstrating a schematic long-axis (longitudinal) section of the device, representing major components, chambers A and B;

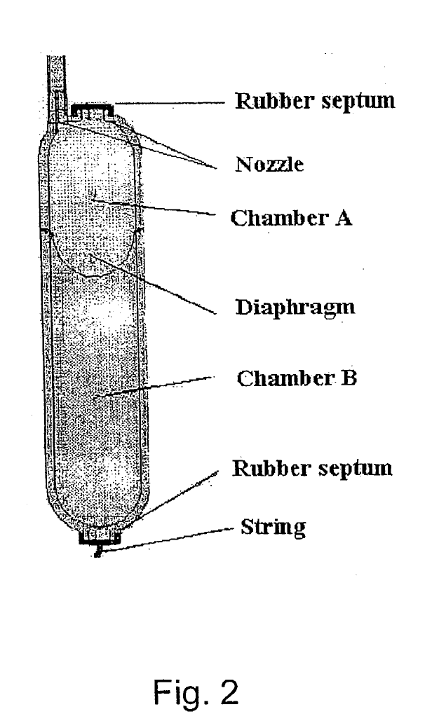

FIG. 3: The device components are schematically presented—as follows:

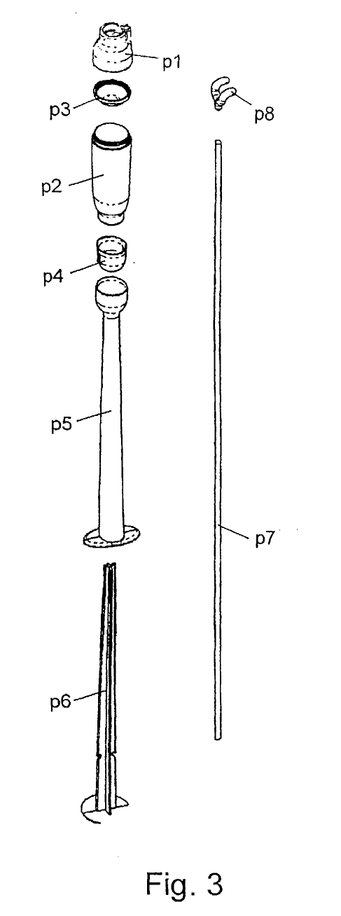

P1: Chamber A containing the fluid preparation intended for delivery into the target organ

P2; Chamber B containing the gas-producing reactant(s),

P3: Elastomeric diaphragm

P4: A cup

P5: A tube

P6: A piston

P7: A stick

P8: “V” guide

FIG. 4: A schematic presentation of injection of the semen sample into chamber A through a rubber septum.

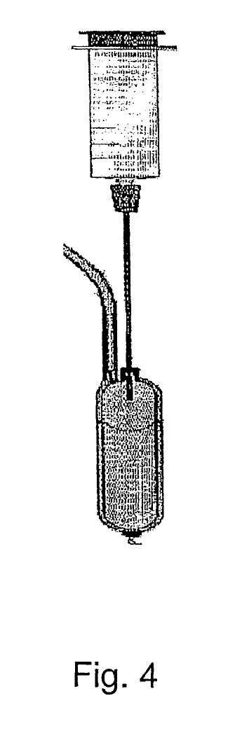

### DETAILED DESCRIPTION OF PREFERRED EMBODIMENTS

The following example is provided merely to illustrate the invention and is not intended to limit the scope of the invention in any manner.

### EXAMPLE

A capsule comprising two coating layers was prepared. The capsule is made of a spherical sugar particle, having a diameter of 1-1.2 mm, which was isolated by a first coating layer (initial coating) containing a mixture of Ethocel (ethyl cellulose) and stearic acid. The coated core is further encapsulated with a second coating layer (main coating) comprising a mixture of calcium carbonate and an inert cellulose derivative(s) such as, for example, Ethocel and Klucel (hydroxypropyl cellulose).

In a preferred embodiment of present invention the initial and main coating layers, each weighs about 10% of the total micro-capsule's weight. Main coating layer consists of 5% (by weight) calcium carbonate (having median particle size of 3 microns), 66.5% Ethocel and 28.5% Klucel.

An individual device containing about 4.5 mg calcium carbonate in an encapsulated form and an amount of 17 mg citric acid. For activating the acid/base reaction, 2-3 ml water are inserted into the device. The water which diffuse into the capsules bring into contact the citric acid and the encapsulated calcium carbonate, as a result a total amount of 1 ml CO₂ is released at a constant rate. The release of CO₂ in chamber B resulting in a 4 hour's constant flow rate of a semen preparation from chamber A into the target organ (vagina).

The flow rate of the liquid preparation from chamber A into the target-organ is controllable by either adjusting the amount of encapsulated reactant and/or increasing/decreasing the thickness and number of capsule's coating layer(s).

1. A sterilizable, compact, portable device for dispensing predetermined amount, at a predetermined flow rate, of a selected liquid preparation into a specific target-organ or tissue in a mammalian body, in particular, of a human, comprising:
(I) a rigid or flexible housing;
(II) an inner, non-stretchable, fluid reservoir chamber (A) containing a predetermined amount of a selected fluid preparation intended for discharging under compression;
(III) an inner, non-stretchable, gas-generating chamber (B) containing at least one ingredient in an encapsulated form, which may react in a reaction yielding a gas; wherein said ingredient is coated by at least one layer;
(IV) an impermeable, stretchable elastomeric diaphragm, separating said chambers (A) and (B); provided that the evolving gas in chamber (B) exerting a pressure on the expansible diaphragm, which compresses the fluid reservoir compartment (A), resulted in expelling the fluid content from chamber (A);
(V) a tubule, a conduit or a hollow needle having an inner end which communicates wit chamber (A) and an outer end which projects outwardly of the housing a short distance, used for delivering the expelled liquid from the reservoir chamber (A) into the target-organ or tissue in the mammal body;
(VI) inlets for inserting the fluid preparation into chamber (A) and water, or other solvent, into chamber (B)
(VII) optionally accessories for assisting in placing, and/or fixing, the device in right position within, or in adjacent to, the desired organ.

2. A device according to claim 1, wherein a fluid preparation having biological or physiological activity is placed in chamber (A).

3. A device according to claim 2, wherein the fluid preparation is a medicine.

4. A device according to claim 2, wherein the fluid preparation is semen

5. A device according to claim 4, wherein the semen is human semen.

6. A device according to claim 1, wherein the device is for a single use (disposable).

7. A device according to claim 1, wherein chamber (B) containing at least one ingredient in an encapsulated form, and said ingredient is coated by two layers; wherein first layer comprising a core being surrounded by a water-insoluble or relatively water-insoluble coating material, and the second layer comprising at least one ingredient in a particulate form which may react in a reaction yielding a gas, being embedded in, or coated by, one or more relatively water-insoluble hydrophilic particulate materials.

8. An encapsulated ingredient, according to claim 7, wherein both coating layers comprising same or different particulate cellulose derivatives.

9. An encapsulated ingredient, according to any of claims 7 and 8, wherein first layer comprising a core being coated by a cellulose derivative polymer and a carboxylic acid of the general formula CH₃(CH₂)ₙCOOH in which n>14.

10. An encapsulated ingredient according to claim 9, wherein the cellulose derivative is ethylcellulose and the carboxylic acid is stearic acid.

11. An encapsulated ingredient according to claim 8, wherein second coating layer consisting of a mixture of ethyl cellulose and hydroxypropyl cellulose.

12. An encapsulated ingredient according to any of claims 1 to 11, wherein the ingredient in a particulate form, which may react in a reaction, yielding a gas is a compound having a carbonate group.

13. An encapsulated ingredient according to claim 11, wherein the carbonate compound is calcium carbonate.

14. An encapsulated ingredient according to claim 7, wherein the core of first layer consists of sugar.

15. A gas-generating chamber (B), according to claim 1, containing one or more particulate ingredients which may react in a reaction yielding a gas, wherein at least one of sad ingredients is in a encapsulated form and coated by at least one layer.

16. A gas-generating chamber (B), according to claim 15 containing a carbonate compound in an encapsulated form and a water-soluble carboxylic acid in a particulate form; wherein said carbonate compound is coated by at least one layer.

17. A gas-generating chamber (B), according to claim 16 containing calcium carbonate in an encapsulated form and citric acid in a participate form; wherein said calcium carbonate is coated by at least one layer.

18. A device according to claim 1, having a cylindrical shape

19. A device according to claim 1 made of non-toxic sterilizable rigid polymeric materials

20. A device according to claims 4 and 5, applicable in a controllable artificial insemination and in carrying out controlled fertilization.

21. A device according to claim 20, comprising in addition a cup attached to chamber (B) to which a tube and a piston is connected assisting in administration of the device into the vagina and placing it in a desired position.

22. A device according to claims 20 and 21 comprising additional accessories and means for retaining said device in the desired position in the patient's vagina or in the patient's uterine cavity.

23. A device according to claim 22 comprising a stick and a guide

24. A device according to claim 19 made of transparent or opaque polymeric materials

25. A device according to claims 1 and 19, wherein chambers (A) and (B) are made of polyethylene and the diaphragm is made of thermoplastic elastomer.

26. A device according to claim 21, wherein the cup is made of polyacetate, the tube is made of polyethylene and the piston is made of high-density polyethylene.

27. A device according to claim 23, wherein the stick is made of high-density polyethylene and the guide is made of thermoplastic elastomer

28. An apparatus for a prolonged-release delivery of a selected fluid preparation into a patient's uterine cavity.

29. A device according to any of the preceding claims in a medical infusion kit form

30. A method for dispensing into a specific targeted organ or tissue in a mammalian body, and in particular in a human, a predetermined amount of a selected liquid preparation, at a predetermined flow rate comprising:
(I) inserting a desired amount of liquid preparation into the chamber (A) of a device according to claim 1;
(II) inserting water, or other solvent, to chamber (B) for actuating the reaction yielding a gas;
(III) placing the device in the desired location and position to allow the liquid preparation to flow from chamber (A) to the desired organ or tissue
(IV) removing the device after completion of the treatment

31. A method according to claim 30 for insemination and fertilization of a human patient.

32. A method according to claim 31, wherein water is added to chamber B for actuation of a reaction between the encapsulated and coated calcium carbonate and water-dissolved citric acid generating a carbon dioxide gas.
